(19) United States
(12) Patent Application Publication
(10) Pub. No.: US 2026/0335130 A1
(43) Pub. Date: Oct. 29, 2026

(54) EYE DROP DISPENSER

(71) Applicant: Aptar Radolfzell GmbH, Radolfzell (DE)

(72) Inventor: Jürgen GREINER-PERTH, Gottmadingen (DE)

(21) Appl. No.: 19/119,549

(22) PCT Filed: Oct. 10, 2023
(86) PCT No.: PCT/EP2023/078041

§ 371 Date: Apr. 9, 2025

(30) Foreign Application Priority Data
Oct. 10, 2022 (EP) 22200655.3

(51) Int. Cl. A61F 9/00; B05B 11/10

(52) U.S. Cl. CPC A61F 9/0008; B05B 11/1016; B05B 11/1047; B05B 11/107

(57) ABSTRACT

An eye drop dispenser for instilling pharmaceutical liquids into an eye of a user. The eye drop dispenser has a liquid reservoir, a discharge opening, a pump device, via which liquid is conveyed out of the liquid reservoir to the discharge opening. The eye drop dispenser has two subunits movable in translation relative to each other and which, for the purpose of actuating the pump device and for effecting discharge of a drop of liquid, can be pressed towards each other in an actuating direction. The discharge opening is oriented such that a discharge of liquid takes place in a discharging direction, which deviates from the actuating direction and encloses an angle of between 45 °and 135 °with the actuating direction.

The invention relates to the field of eye drop dispensers.

Eye drop dispensers are used for the administration of fluid to the eye of a user. In particular, the fluid may be a fluid for treatment of dry or red eyes. However, other medications, such as, for example, antibiotics, can be dispensed by means of generic eye drop dispensers and eye drop dispensers according to the invention.

The most conventional way of administering eye drops involves allowing the eye drops to fall from above into the eye by means of an eye drop dispenser. From the eye drop dispenser, for example, a squeeze bottle dispenser, a quantity of fluid which first accumulates on a drop formation face of the dispenser and is released therefrom when it becomes sufficiently large and falls as intended into the eye of the user is carefully dispensed.

The use of such a conventional eye drop dispenser involves various problems. On the one hand, it is difficult for many users to hit the eye. When looking upward as necessary, it cannot be easily estimated when the discharge opening of the dispenser is located directly above the eye. This can also lead to the eye drop dispenser being moved very close to the eye by the user, which can result in contact and pain. The generally very large droplet striking the eye is also perceived to be unpleasant by many users.

### PROBLEM AND SOLUTION

An object of the invention is to provide an eye drop dispenser and an operating method therefor which with regard to user comfort and/or administration precision are superior to known eye drop dispensers.

In order to achieve this objective, an eye drop dispenser for administration of pharmaceutical fluids to an eye of a user is proposed and is configured as follows.

The eye drop dispenser has a fluid store which in the state ready for sale is filled with a pharmaceutical fluid for administration to an eye of the user. This may in particular be a pharmaceutical fluid for the treatment of increased internal eye pressure (glaucoma treatment) or for treating dry eyes and for treating allergies and inflammations. In this instance, the molecular groups alpha-2 agonists, for example, brimonidine, prostaglandin analogs (tafluprost, latanoprost, bimatoprost, travoprost), beta-blockers, for example, timolol, and carbonic anhydrase inhibitors, for example, dorzolamide or hyaluronic acid compounds, film formers, for example, methyl cellulose compounds and cyclosporine or antihistamines, for example, olopatadine and levocabastine, steroids, for example, loteprednol and dexamethasone, and NSAIDS, for example, ketorolac, are significant. As already mentioned in the introduction, these may also be other medications, such as, for example, an antibiotic.

The eye drop dispenser according to the invention further has a discharge opening through which the fluid can be discharged, wherein the discharge through this discharge opening is preferably carried out substantially in a horizontal direction. A drop which is discharged through the discharge opening is virtually propelled into the eye.

In order to convey the fluid from the fluid store to the discharge opening, there is provided a pump device which in particular is provided for manual activation. It may particularly be a piston pump, the activation of which reduces a pump chamber so that the fluid which is contained in the pump chamber and which has previously been drawn from the fluid store is placed under pressure and is discharged in the direction of and out through the discharge opening.

In order to activate the pump device, the eye drop dispenser has two part-units which can be moved relative to each other in translation and which in order to activate the pump device and consequently to bring about a discharge of a fluid drop can be pressed toward each other in an activation direction. This activation is preferably carried out counter to the force of a resilient device, in particular a helical spring which acts between the part-units. Preferably, this involves a main unit which during use is gripped by a hand of the user and an activation unit which is activated using a finger of the hand, in particular pressed down.

Preferably, one of the two part-units contains the fluid store and the other of the two part-units has the discharge opening. However, configurations are also possible in which the discharge opening and the fluid store are provided on the same part-unit. The other part-unit is then substantially only an activation pressing device for activating the pump device.

As already mentioned above, with an eye drop dispenser according to the invention there is provision for the fluid in the form of a coherent defined quantity of fluid, referred to in the context of the invention as a drop, to be virtually propelled into the eye, generally with a substantially horizontal orientation. The activation of the eye drop dispenser is, however, carried out in a different direction from this. This is achieved by the discharge opening being orientated in such a manner that a fluid discharge is carried out in a discharge direction which differs from the activation direction of the part-units. Preferably, the activation direction and the discharge direction enclose an angle between 45 ° and 135 °, in particular approximately or precisely an angle of 90 °. The activation direction which is angled with respect to the discharge direction in the manner mentioned preferably also corresponds to the main extent direction, which is defined by a center axis of the fluid store, of the eye drop dispenser.

An eye drop dispenser according to the invention is particularly pleasant to handle. The activation is carried out by the pump device being activated by pressing together the part-units substantially vertically in practice, wherein the fluid drop is discharged transversely relative thereto and propelled into the eye.

So that the fluid striking the eye is not unpleasant for the user or ideally is not noticeable, it is proposed to configure the pump device in such a manner that, with complete activation, that is to say, with a displacement of the part-units from a remote end position into a close end position, a fluid volume of less than 25 μl is conveyed in the direction of the discharge opening. Preferably, the volume of fluid is even lower and is particularly preferably below 20 μl. This quantity of fluid then forms during discharge a fluid drop. The volume of less than 25 μl, in particular of less than 20 μl, is less than the drop volume which is generally achieved via a dispenser for discharging falling drops. However, it has been found that in many applications, particularly when a complete wetting of the eye is desired, a volume of 25 μl is sufficient, particularly since the quantity of fluid as a result of the administration precision which can be achieved with the eye drop dispenser according to the invention reaches the eye completely and not only partially.

The small volume of the fluid drop in the context of the propelled drop which is provided in this instance is useful to make the striking of the fluid in the eye not unpleasant for the user.

The mentioned small pump volume of the pump device per activation travel may be problematic with regard to the ventilation of a pump chamber of the pump device when the dispenser is started up for the first time, the so-called “priming”. A small pump volume leads to the excess pressure produced by means of activation of the air located in the pump chamber in the delivery state not being sufficient to open an outlet valve which opens dependent on pressure. In order to overcome this problem, it is advantageous for a pump device to be provided with a ventilation function. With such a pump device with a ventilation function, toward the end of the activation travel, that is to say, when the two part-units have been moved toward each other to the maximum extent, a communicating connection between the fluid store and pump chamber is opened. Via this connection, the compressed air can at least partially escape from the pump chamber so that during the subsequent return travel incompressible fluid is drawn into the pump chamber. Repeated activations lead to the air being successively completely displaced from the pump chamber.

In addition to the volume, the speed at which the drop is discharged and with which it strikes the eye is also relevant for the question as to whether the drop striking the eye is perceived to be unpleasant. The dispenser is preferably configured to discharge the fluid with an activation speed of 35 mm/sec at a speed of at least 2 m/sec, in particular at a speed of at least 3 m/sec, so that the flight path of the drop is substantially linear. At the same time, the speed is preferably no more than 8 m/sec and preferably below 6 m/sec in order not to perceive the striking against the eye to be unpleasant or in an ideal case not to be able to notice it at all or only slightly.

The mentioned speeds are substantially below normal discharge speeds of fluid dispensers. For example, the discharge speed of nasal dispensers is generally in the range between 10 m/sec and 20 m/sec.

In addition to the volume of the drop and the period of time over which this volume is discharged, the configuration of the discharge opening is also relevant for the discharge speed. For an eye drop dispenser of the type according to the invention, it is preferable for the discharge opening to have a comparatively large cross sectional surface-area at the narrowest location thereof. The clear cross section at that location is preferably at least 0.2mm² , particularly preferably 0.5 mm² or more. Preferably, the discharge opening is circular and has a diameter between 0.5 mm and 0.8 mm.

The geometry of the discharge opening and the direct supply to the discharge opening is preferably configured in such a manner that the fluid does not develop any swirling motion with respect to the axis of the discharge direction since such a swirling motion could result in the undesirable atomization of the fluid. Preferably, the supply is carried out through at least one radial supply channel, particularly preferably through at least two supply channels which are distributed evenly over the circumference in order to enable a swirl-free supply.

The avoidance of a swirling motion and a corresponding tendency to atomization of the fluid can also be achieved by the fluid itself. It has been found that fluid with a viscosity which is higher than water is particularly suitable for being propelled in the form of a drop. Preferably, the fluid store is therefore filled with a fluid with a viscosity of at least 2 cP, preferably with a viscosity between 10 cP and 80 cp, in particular between 30 cP and 50 cP.

In order to produce a drop, which is discharged in a shot-like manner, of the desired volume, it is considered to be advantageous for the part-travel, which is active for the discharge, of the pump device, that is to say, the part-travel of the part-units upon activation, during the passage of which the discharge takes place, to preferably be a maximum of 2 mm, particularly preferably 1.5 mm or less. In the case of a piston pump as a pump device, the volume of the drop is the product of this part-travel and the effective piston surface-area. The comparatively small active part-travel ensures a rapid passage and the defined form, which can thereby be achieved, of the propelled drop.

A particularly preferred configuration of the pump device makes provision for it to be configured, during the displacement of the two part-units from a non-activated idle position to first pass through an idle travel path and subsequently an active travel path. During the displacement of the part-units over the idle travel path, there is no application of pressure to the fluid in the pump chamber of the pump device. Preferably, the fluid is displaced from the pump chamber whilst passing through the idle travel path back into the fluid store. Only when the part-units have been moved together over the idle travel path does the active travel path follow, over which the reduction of the pump chamber which is then isolated with respect to the fluid store and resulting from this the discharge of the fluid in the form of a propelled drop take place.

The idle travel path mentioned is not necessarily important for the function of the eye drop dispenser. The discharge of the fluid can also begin directly with the beginning of the displacement of the two part-units. However, it has been found that the operation of users is perceived to be significantly more pleasant when over a part-travel, the mentioned idle travel path, no discharge is carried out so that during the positioning of the eye drop dispenser there is no risk of an unintentional discharge. In addition, the idle travel path leads to it being easier to pass through the subsequent active travel path with an appropriately high displacement speed of the part-units.

The required manual activation force during the fluid discharge is with respect to an activation speed of 35 mm/sec preferably at least 15 newtons, in particular at least 18 newtons. This force is determined by the restoring spring of the eye drop dispenser and the flow resistance to which the fluid is subjected between the pump chamber and discharge opening and can in particular also be caused by a valve which is located in the flow path and the required opening pressure thereof.

As long as an idle travel path which is upstream of the active travel path is provided, the activation force required at that location is smaller, primarily as a result of the fact that the fluid in this phase does not yet flow and consequently the flow resistance does not influence the activation force. The activation force during the passage of the idle travel path is with respect to an activation speed of 35 mm/sec preferably below 15 newtons, in particular preferably below 12 newtons.

The difference in the activation force between the idle travel path and active travel path is preferably with a conventional activation speed of 35 mm/sec at least 3 newtons, preferably at least 5 newtons. This brings about the haptic effect of a pressure point. When displacing the part-units, the beginning of the active travel path can be simply perceived. Preferably, the transition between the idle travel path and the active travel path is configured in such a manner that the required activation force increases over a path of at least 0.3 mm so that the haptic localization of the change is facilitated.

A conventional procedure during use is that the user positions the dispenser in front of the eye, carefully applies force manually to the part-units until he/she has located the transition from the idle travel path and active travel path, spaces the part-units slightly apart from each other again and then presses the part-units together with a defined and sufficiently high force so that the idle travel path and active travel path are passed through in rapid succession.

The length of the idle travel path is preferably more than 1 mm, in particular at least 3 mm or even at least 4 mm. The active travel path is preferably shorter than the idle travel path. Preferably, the active travel path is a maximum of 2 mm, particularly preferably a maximum of 1.5 mm.

The active travel path can end with a stop-like delimitation, over which the part-units cannot be moved closer together. However, it is considered to be advantageous for the active travel path to be adjoined by another residual travel path. When the residual travel path is reached, the discharge ends since the excess pressure in the pressure chamber is cancelled, particularly preferably by a communicating connection being provided between the pump chamber and the fluid store so that fluid with a pump chamber which is continuously reduced over the residual travel path can flow back into the fluid store.

In particular, the residual travel path mentioned may be advantageous during the start-up of the dispenser and the so-called “priming”, that is to say, the initial displacement of air from the pump chamber, since, with an almost completely compressed pump chamber, the air which is compressed therein is substantially completely discharged from the pump chamber into the fluid store. In addition, the residual travel path improves the metering precision since the metering precision is not impaired by a potentially inadequately defined stop at the end of the active travel path or an involuntary braking of the manual activation by the user as a result of the approaching stop.

In order to technically implement an idle travel path and a residual travel path, there may, for example, be provision for a piston of the piston pump to be movably provided in the part-unit which is associated therewith so that via the idle travel path initially a displacement of the piston with respect to this part-unit is carried out before subsequently after reaching an end position the mentioned part-unit together with the piston is displaced with respect to the other part-unit and the piston cylinder which is provided thereon.

However, another configuration is more advantageous in which the pump device has an inlet valve which is in the form of a sliding valve. Such a sliding valve is characterized in that there is provided a valve slide which at the beginning of the activation is arranged outside a metering channel and during the activation is introduced into the metering channel and displaced therein. The sliding valve preferably forms in this instance an inlet valve of the pump device. Until the valve slide together with the metering channel closes the inlet, the pump chamber is connected to the fluid store. As soon as the valve slide is introduced into the metering channel, the pump inlet is closed and the continued activation leads to the pressure increase in the fluid in the pump chamber and to the fluid discharge. The inlet into the metering channel is preferably provided with a conical introduction face which in the activation direction extends at least over 0.3 mm. This leads to a gentle capture of the valve slide and a good haptic detectability of the change from idle travel to the active travel.

Preferably, the components are sized in such a manner that the valve slide at the end of the activation slides out of the metering channel again, that is to say, again at least partially loses contact with the wall of the metering channel so that the pump chamber and the fluid store are thereby connected. The part-path of the displacement beyond the metering channel forms the residual travel path described above with regard to its action and the ventilation function mentioned in the introduction.

The valve slide forms a type of piston in the metering channel. In order to reduce the pump chamber volume, however, there is preferably provided an additional larger pump piston which is fixed in position with respect to the valve slide and, at least over the active travel path, but preferably permanently, bears on an associated pump cylinder wall which in turn is provided in a fixed manner with respect to the metering channel. The reverse configuration is also possible, that is to say, a fixed state of the valve slide and pump cylinder wall with respect to each other and a fixed state of the metering channel and pump piston, on the other hand.

Preferably, a valve may be provided between the pump chamber and the discharge opening. In particular in this instance there is preferably provided a pump outlet valve which closes at reduced pressure in the pump so that during the return travel fluid is drawn from the fluid store into the pump chamber.

In a further preferred manner, the eye drop dispenser has a discharge valve which opens in a manner dependent on pressure and which is arranged directly upstream of the discharge opening. In this instance, this may be an additional valve in addition to a pump outlet valve. The discharge valve may, however, also be the only valve between the pump chamber and the discharge opening.

The discharge valve opens with a defined minimum pressure and closes as soon as the fluid pressure of the incoming fluid falls below this minimum pressure. As a result of the arrangement of the discharge valve directly in front of the discharge opening, a very defined beginning of the discharge and a very defined end of the discharge is brought about, wherein this is advantageous for forming the propelled drop. Preferably, the discharge valve is constructed to open at a reduced pressure in the fluid of a maximum of 6 bar, preferably at a reduced pressure in the fluid of a maximum of 4 bar. These comparatively small opening pressures are sufficient for the formation of a propelled drop. The pressure required to completely open the discharge valve is preferably only slightly above this minimum opening pressure, particularly preferably no more than 1 bar above the minimum opening pressure.

The discharge valve is preferably provided with a resilient device, by means of which a valve member of the discharge valve is pressed into a closed position. In particular, the configuration of this spring determines the mentioned opening pressure.

The discharge opening of the eye drop dispenser according to the invention is according to the invention configured for discharge in a discharge direction which is angled with respect to the activation direction. Preferably in this instance, a right-angle is provided. The eye drop dispenser may be configured in such a manner that, with respect to the axis of the activation direction, it has a substantially rotationally symmetrical shaping. The discharge opening in such a case does not protrude laterally beyond the covering face of the respective part-unit.

However, it is also conceivable to arrange the discharge opening at the end of a radially outwardly extending discharge nozzle, preferably a discharge nozzle which is configured integrally with an activation face of the part-unit.

A radially outwardly extending discharge nozzle forms a good auxiliary orientation member in order to be able to grip the dispenser directly in the correct orientation or to be able to orientate it in a simple manner after gripping. In particular, an outer face of the discharge nozzle is preferably circular and concentric with the discharge opening.

As an additional measure to facilitate the orientation, there may be provision for the discharge opening to be able to be easily detected visually, in particular by means of a color contrast. The discharge opening is located during correct use of the eye drop dispenser preferably between 2 cm and 6 cm away from the eye so that focusing may be difficult. In order to facilitate the orientation, there may in particular be provision for a color contrast between the component which forms the discharge opening and a valve member of the discharge valve to be provided. Preferably the valve member is darker than the discharge opening member. In a particularly preferred manner, the valve member has a color which is significantly different from white, such as blue, red, green or purple.

A preferred construction type of the eye drop dispenser according to the invention makes provision for the fluid store to face downward during use and for the pump device to be arranged above the fluid store. In order to draw the fluid from the fluid store, in this instance a riser pipe which protrudes from the pump device into the fluid store is preferably provided. With this construction type, the second part-unit is during use preferably provided above the pump device and forms the activation unit which is pressed down with respect to the first part-unit comprising the fluid store. The discharge opening is preferably provided on the second part-unit. For activation, the first part-unit which virtually forms the main part-unit is gripped with the hand and the second part-unit, the activation unit, is pressed down using a finger of this hand.

Another variant makes provision for the fluid store to be provided above the pump device during use and to form a portion of the second part-unit, wherein in this instance this second part-unit is pressed down with respect to the first part-unit which is located below it and the fluid is discharged from the discharge opening of this first part-unit. Also in this instance, the first part-unit is preferably the main part-unit which for the purposes of discharge is gripped with a hand, whilst the second part-unit forms the activation unit which is pressed down by means of a finger.

In addition to the eye drop dispenser itself, the invention also relates to an operating method therefor.

According to this method, for the purposes of discharge, the two part-units are moved in an activation direction toward each other from a non-activated idle position, whereby fluid is acted on with pressure by means of a pump device and fluid is thereby discharged through a discharge opening in a discharge direction which differs from the activation direction. In this instance, the fluid discharge is carried out in the form of a propelled drop with a drop volume between 5 μl and 25 μl which is discharged at a speed between 3 m/sec and 8 m/sec.

In particular, there may be provision for the two part-units to be moved toward each other starting from a non-activated idle position, wherein initially over an idle travel path of preferably at least 1 mm a pump chamber of the eye drop dispenser is connected in a communicating manner to the fluid store or is not reduced and wherein subsequently over an active travel path which follows the idle travel path of preferably less than 2 mm the pump chamber is reduced, whilst it is no longer connected to the fluid store in a communicating manner so that the fluid pressure in the pump chamber is increased and the discharge of the drop is brought about.

### BRIEF DESCRIPTION OF THE DRAWINGS

Other advantages and aspects of the invention will be appreciated from the claims and the following description of a preferred exemplary embodiment of the invention which will be explained below with reference to the Figures. In the drawings:

FIG. 1 shows an eye drop dispenser according to the invention as a sectioned illustration.

FIGS. 2A to 2D show the eye drop dispenser in various phases during the activation.

FIG. 3 shows the discharge opening of the eye drop dispenser and the geometry of the wall at the inner side of the discharge opening.

### DETAILED DESCRIPTION OF THE EXEMPLARY EMBODIMENTS

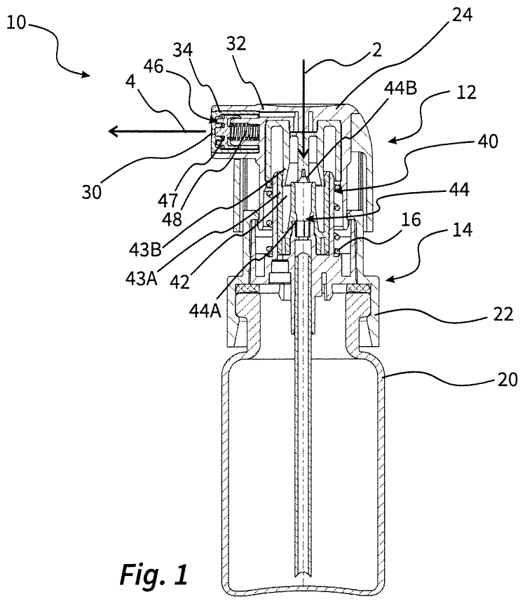

FIG. 1 shows a drop dispenser 10 according to the invention. The drop dispenser 10 has a fluid store 20 and a discharge head which is fitted to the fluid store 20 and which has a base 22 and an activation pressing member 24.

The components mentioned form two part-units 12, 14 which can be displaced with respect to each other, wherein the base 22 of the discharge head and the fluid store 20 form a main part-unit 14 and wherein the activation pressing member 24 forms an activation unit 12.

The eye drop dispenser 10 has a pump device 40 which is in the form of a piston pump and which is arranged between the part-units 12, 14 so that by pressing together the part-units 12, 14 pressure can be applied to fluid in a pump chamber 42 in order to thereby be able to discharge fluid through the discharge opening 30 on the part-unit 12.

The pump device 40 has an inlet valve 44 which is in the form of a sliding valve. This valve has a metering channel 44A which is provided on the base 22 of the discharge head and a valve slide 44B which is provided on the activation pressing member 24.

The pump device 40 further has a pump outlet valve which in this instance is formed by a discharge valve 46 which is arranged directly upstream of the discharge opening 30. This discharge valve 46 comprises a valve member 47 which can be moved with respect to a valve seat and which is pressed by a valve spring 48 into the closed position.

The part-units 12, 14 are acted on with force with respect to each other by means of a restoring spring 16. In the idle position of FIG. 1, they take up their maximum spacing with respect to each other. In this idle position, the valve slide 44B of the inlet valve 44 is not yet arranged in the metering channel 44A. The fluid store 20 and the pump chamber 42 are accordingly in this initial state still connected to each other in a communicating manner.

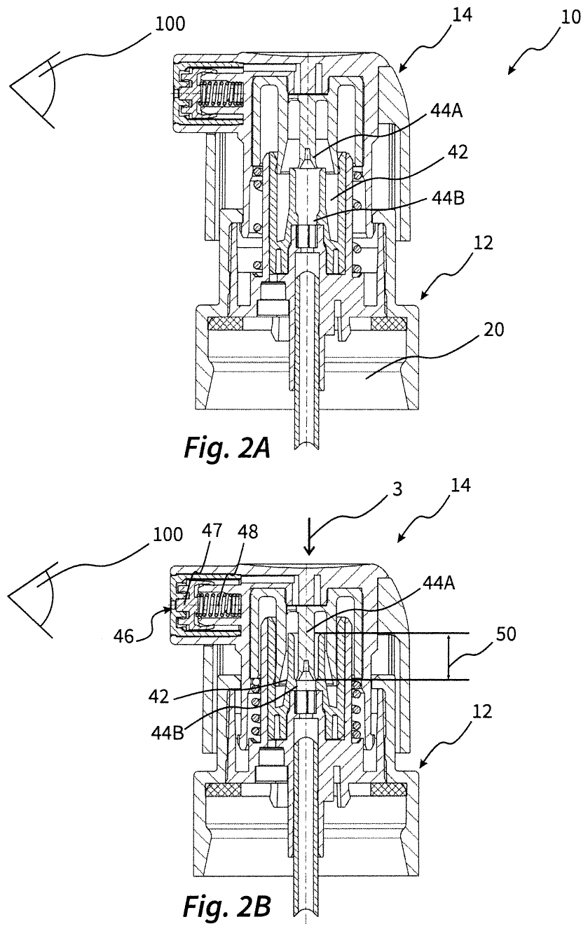

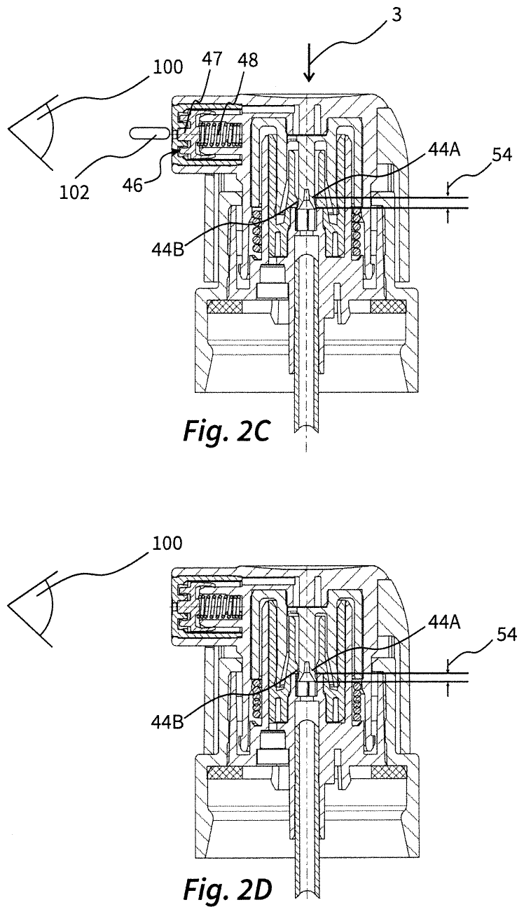

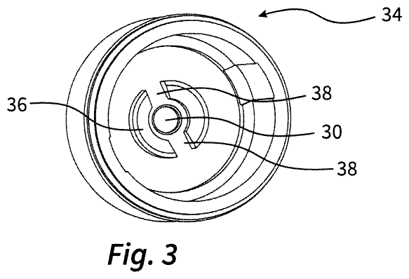

With reference to FIGS. 2A to 2D, the operating method of the eye drop dispenser 10 is explained.

FIG. 2A shows again the idle state of FIG. 1. In addition, it can be seen from this Figure that the eye drop dispenser 10 for correct discharge of eye drops is positioned at the height of the eye 100 and approximately from 3 to 5 cm away from it.

Starting from this position and under the assumption that the pump chamber 42 at this time is already completely filled with fluid, an activation in the direction of the arrow 3, see transition from FIG. 2A to FIG. 2B, initially does not lead to a discharge of fluid. Although the part-unit 14 is pressed down or the part-unit 12 is raised, the valve slide 44B in this phase does not yet come into contact with the wall of the metering channel 44A. In this phase of the activation, therefore, fluid from the pump chamber 42 can still flow back into the fluid store 20 during the reduction of the inner volume of the pump chamber 42. Between the position of FIG. 2A and the position of FIG. 2B, in which the part-unit 12 is pressed down substantially by 4 mm, the user can move the part-units freely with respect to each other without this having any effects on a fluid discharge. This also enables him/her to precisely locate the intermediate position of FIG. 2B in which the valve slide 44B is in contact with the metering channel 44A. The part-path along which the part-units 12, 14 can be moved with respect to each other without any discharge effect represents the so-called idle travel path 50.

As soon as the eye drop dispenser 10 is positioned correctly with respect to the eye 100 and the user has gained an impression of the position of the part-units 12, 14 in the stage of FIG. 2B, the user presses the part-unit 14 down as far as the position of FIG. 2C. In this instance, in the position of FIG. 2B the valve slide 44B initially seals with the metering channel 44A, whereby the pump chamber 42 is isolated with respect to the fluid store. The continued movement along the active travel illustrated in FIG. 2C now leads to the pressure in the pump chamber 42 increasing as a result of the incompressibility of the fluid and consequently the discharge valve 46 opens by the valve member 47 being pressed to the right counter to the force of the valve spring 48 with respect to FIG. 2C. As can be seen with reference to FIG. 2C, this leads to a shot-like discharge of a drop 102. The drop 102 is discharged horizontally without atomization approximately at a speed of 1.5 m/s and strikes the eye 100 immediately afterwards. As a result of the horizontal and linear movement direction of the drop 102, this is very precise.

As soon as the valve slide 44B at the lower end leaves the metering channel 44A, illustrated by the state of FIG. 2C, a communication between the pump chamber 42 and the fluid store 20 past the valve slide 44B is possible again. Suddenly, the excess pressure in the pump chamber 42 collapses and the discharge valve 46 closes in an equally sudden manner. This leads to a precise breakaway of the fluid drop 102 from the discharge opening 30. As a result of the sudden breakaway of the fluid drop 102, the retention of residual fluid outside the discharge opening 30 is also effectively prevented.

If after activation the force applied to the part-units 12, 14 finally subsides, the part-units 12, 14 return under the action of the restoring spring 16 into their original position. Whilst the valve slide 44B in this instance passes through the metering channel 44A again, the supply into the pump chamber 42 is briefly interrupted. A reduced pressure is briefly formed in the pump chamber 42 in this instance. As soon as the valve slide 44B leaves the metering channel 44A again at the upper end and the communicating connection with respect to the fluid store 20 is produced again, fluid is drawn in and fills the pump chamber 42.

FIG. 3 shows a housing component 34 in which the discharge opening 30 is provided. It can be seen that the discharge opening is comparatively large, in this instance preferably has a diameter between 0.5 and 0.8 mm. This relatively large diameter serves not to accelerate the discharged fluid in an excessively powerful manner and in addition to avoid atomization.

Also for the purposes of avoiding atomization, there is provision for the fluid to reach the discharge opening 30 through two supply openings 38 in an internal web 36 which surrounds the discharge opening 30, wherein the supply openings 38 are in each case radially orientated and arranged opposite each other. In this manner, a state in which the fluid is not atomized during discharge is also promoted.

1. An eye drop dispenser for administration of pharmaceutical fluids to an eye of a user, comprising:
a fluid store;
a discharge opening;
a pump device conveying fluid from the fluid store to the discharge opening; and
two part-units movable in translation with respect to each other, wherein the two-part units are pressed in an activation direction toward each other to activate the pump device and to bring about a discharge of a fluid drop;
wherein the discharge opening is configured such that a fluid discharge is carried out in a discharge direction differing from the activation direction.

2. The eye drop dispenser as claimed in claim 1, wherein:
the pump device is in the form of a piston pump and has a pump chamber and by displacing the two part-units the pump chamber is reduced during an actuation travel to apply pressure to fluid located in the pump chamber.

3. The eye drop dispenser as claimed in claim 1, wherein
the pump device has a pump volume per actuation travel of less than 25 μl.

4. The eye drop dispenser as claimed in claim 1, wherein
the discharge opening has a clear cross section of at least 0.2 mm².

5. The eye drop dispenser as claimed in claim 1,
wherein the discharge opening is associated with at least one supply channel extending radially with respect to the discharge direction and the at least one supply channel supplies fluid in a swirl-free manner to the discharge opening.

6. The eye drop dispenser as claimed in claim 1, wherein
the pump device is configured such that, during displacement of the two part-units from a non-activated idle position, first an idle travel path and subsequently an active travel path are passed through;
during displacement of the part-units over the idle travel path no pressure is applied to the fluid in a pump chamber of the pump device; and
during the displacement of the part-units over the active travel path fluid pressure in the pump chamber rises and fluid is discharged from the pump chamber in a direction of the discharge opening.

7. The eye drop dispenser as claimed in claim 6, wherein
an activation force intended to be applied to press together the part-units over the idle travel path is below 15 newtons and an activation force at a transition between the idle travel path and the active travel path is above 15 newtons.

8. The eye drop dispenser as claimed in claim 1 wherein:
the pump device has an inlet valve in the form of a sliding valve, wherein the sliding valve has a metering path in the form of a cylindrical metering channel and a valve slide closing a pump chamber at the inlet side by the valve slide being introduced into the metering channel; and
in a non-activated idle position of the two part-units, the valve slide is located outside the metering channel and moves during activation into the metering channel at an inlet side.

9. The eye drop dispenser as claimed in claim 8, wherein:
the pump chamber is delimited by a pump cylinder and the pump has a pump piston movable in the pump cylinder;
the valve slide of the inlet valve is provided in a fixed manner with respect to the pump piston; and
the cylindrical metering channel of the inlet valve is provided in a fixed manner with respect to the pump cylinder.

10. The eye drop dispenser as claimed in claim 1, wherein
the eye drop dispenser has a discharge valve, the discharge valve opening in a manner dependent on pressure and being is arranged directly upstream of the discharge opening.

11. The eye drop dispenser as claimed in claim 1, wherein:
a first of the two part-units forms a main part-unit, the main part-unit for discharge is gripped with a hand; and
a second of the two part-units forms an activation unit, the activation unit at an end front face having an actuation face pressable downwardly with the of the finger of the hand.

12. The eye drop dispenser as claimed in claim 11, wherein
the discharge opening is provided on the second of the two part-units.

13. The eye drop dispenser as claimed in claim 1, wherein
the eye drop dispenser has a substantially rotationally symmetrical outer contour, and in a region of the discharge opening a radially outwardly extending discharge nozzle having a distal end is disposed and the discharge opening is provided at the distal end of the discharge nozzle.

14. The eye drop dispenser as claimed in claim 2, wherein
the pump device has a ventilation function and toward the end of an activation travel a communicating connection is provided by the ventilation function between the fluid store and the pump chamber.

15. The eye drop dispenser as claimed in claim 8, wherein
before reaching an activated end position, the valve slide moves at an outlet side out of the metering channel.

16. The eye drop dispenser as claimed in claim 10, wherein
the discharge valve of the eye drop dispenser is formed by a pump outlet valve, the pump outlet valve delimiting a pump chamber of the pump at an outlet side.

17. The eye drop dispenser as claimed in claim 10, wherein
the discharge valve has a resilient device, the resilient device pressing a valve member into a closure position, and the discharge valve is configured such that a fluid pressure of less than 6 bar opens the discharge valve.

18. The eye drop dispenser as claimed in claim 11,
wherein the pump device is provided with a riser pipe, the riser pipe protruding into the fluid store.

19. The eye drop dispenser as claimed in claim 1, wherein
the discharge opening is provided on a first of the two part-units.

20. The eye drop dispenser as claimed in claim 1, wherein
the discharge opening is highlighted by a color contrast, and a component through which the discharge opening extends has a color differing from a valve member visible through the discharge opening.

21. A method for operating an eye drop dispenser as claimed in claim 1, wherein:
the two part-units are moved toward each other in the activation direction from a non-activated idle position, whereby fluid is acted on with pressure by the pump device and fluid is thereby discharged through the discharge opening in the discharge direction differing from the activation direction; and
fluid discharge is carried out in the form of a propelled drop with a drop volume between 5 μl and 25 μl which is discharged at a speed between 3 m/sec and 8 m/sec.

22. The method as claimed in claim 21,
wherein the two part-units, starting from the non-activated idle position are moved toward each other, wherein initially over an idle travel path a pump chamber of the eye drop dispenser is connected in a communicating manner to the fluid store or is not reduced and wherein subsequently over an active travel path which follows the idle travel path the pump chamber is reduced, whilst the pump chamber is no longer connected to the fluid store in a communicating manner so that fluid pressure in the pump chamber is increased and discharge of a drop is brought about.

23. The method as claimed in claim 22, wherein
after passing through the active travel path by moving the two part-units toward each other, the two part-units reach a ventilation position in which a ventilation of the pump chamber is carried out, by a communicating connection produced in the ventilation position between the fluid store and the pump chamber.

24. The eye drop dispenser as claimed in claim 1, wherein the discharge direction encloses with the activation direction an angle between 45 ° and 135 °.

25. The eye drop dispenser as claimed in claim 1, wherein the discharge direction encloses with the activation direction an angle between 80 ° and 100 °.
